(19) United States
(12) Patent Application Publication
(10) Pub. No.: US 2006/0019969 A1
(43) Pub. Date: Jan. 26, 2006

(54) Use of compounds active on the sigma receptor for the treatment of allodynia

(75) Inventor: Jose Manuel Baeyens Cabrera, Granada (ES)

Correspondence Address: BANNER & WITCOFF, 1001 G STREET N W, SUITE 1100, WASHINGTON, DC, 20001, US

(73) Assignee: Laboratorios Dr. Esteve S.A., Barcelona (ES)

(21) Appl. No.: 10/902,273

(22) Filed: Jul. 30, 2004

(30) Foreign Application Priority Data
Jul. 24, 2004 (EP) 04017562.2

(51) Int. Cl. A61K 31/519

(52) U.S. Cl. 514/259.41

(57) ABSTRACT

The present invention refers to the use of compounds active on the sigma receptor for the treatment of allodynia.

### FIELD OF THE INVENTION

The present invention refers to the use of compounds active on the sigma receptor for the treatment of the symptoms of allodynia, as well as treatment of the disease causing the symptoms, as well as the prevention of the prophylaxis of the disease causing the symptoms.

### BACKGROUND OF THE INVENTION

The treatment of pain conditions is of great importance in medicine. There is currently a world-wide need fro additional pain therapy. The pressing requirement for a specific treatment of pain conditions or as well a treatment of specific pain conditions which is right for the patient, which is to be understood as the successful and satisfactory treatment of pain for the patients, is documented in large number of scientific works which have recently and over the years appeared in the field of analgesics or on basic research on nociception.

PAIN is defined by the International Association for the Study of Pain (IASP) as “an unpleasant sensory and emotional experience associated with actual or potential tissue damage, or described in terms of such damage (IASP, Classification of chronic pain 2^{nd} Edition, IASP press (2002), 210). Even though pain is always subjective its causes or syndromes can be classified.

Especially allodynia which in the past years has developed into a major health problem in broad areas of the population needs a very specific treatment, especially considering that any treatment of allodynia is extremely sensitive to the causes behind the pain, be it the disease ultimately causing it or the mechanistic pathway over which it develops.

Therefore, it was the underlying problem solved by this invention to find new ways of treating allodynia.

Therefore, it was the underlying problem solved by this invention to find new ways of treating allodynia.

So, the main object of this invention is the use of a compound binding to the sigma receptor in the production of a medicament for the treatment of allodynia.

This/these compound/s may be in neutral form, the form of a base or acid, in the form of a salt, preferably a physiologically acceptable salt, in the form of a solvate or of a polymorph and/or in the form of in the form of its racement, pure stereoisomers, especially enantiomers or diastereomers or in the form of mixtures of stereoisomers, especially enantiomers or diastereomers, in any suitable ratio.

While working on compounds binding to the sigma receptor and with models like knock-out mice it was surprisingly found out that allodynia is connected to the sigma receptor so that compounds binding to the sigma receptor were acting on allodynia with a high potency.

“Treating” or “treatment” as used in this application are defined as including the treatment of the symptoms—of allodynia—as well as treatment of the disease or disease consequences causing the symptoms, the prevention or the prophylaxis of the symptoms—of allodynia—as well as the prevention or the prophylaxis of the disease or disease consequences causing the symptoms. Preferably “treating” or “treatment” as used in this application are defined as including the treatment of the symptoms—of allodynia—as well as treatment of the disease consequences causing the symptoms, the prevention or the prophylaxis of the symptoms—of allodynia—as well as the prevention or the prophylaxis of the disease consequences causing the symptoms. Most preferably “treating” or “treatment” as used in this application are defined as including the treatment of the symptoms of allodynia, and the prevention or the prophylaxis of the symptoms of allodynia.

“The sigma receptor/s” as used in this application is/are well known and defined using the following citation: This binding site represent a typical protein different from opiod, NMDA, dopaminergic, and other known neurotransmitter or hormone receptor families (G. Ronsisvalle et al. Pure Appl. Chem. 73, 1499-1509 (2001)).

Pharmacological data based on ligand binding studies, anatomical distribution and biochemical features distinguish at least two subtypes of σ receptors (R. Quiron et al., Trends Pharmcol. Sci. 13, 85-86 (1992); M. L. Leitner, Eur. J. Pharmacol. 259, 65-69 (1994); S. B. Hellewell and W. D. Bowen; Brian Res. 527, 244-253 (1990)) (G. Ronsisvalle et al. Pure Appl. Chem. 73, 1499-1509 (2001)). The protein sequence of sigma receptors (Sigma 1 (σ1) and Sigma 2 (σ2)) is known (e.g. Prasad, P. D. et al, J. Neurohem. 70 (2), 443-451 (1998)) and they show a very high affinity for e.g. pentazocine. Another selective ligand is a compound known as NE-100 (Chaki, S. et al., Eur. J. Pharmacol. 251, R1-R2 (1994)).

“Compound/s binding to the sigma receptor” as used in the application is/are defined as having ≧95% displacement using 1 mM (¹H-pentazocine) and a Kₘ Value in their binding to the sigma receptor ≦50 nM (in regards to any one of the sigma receptor subtypes).

Compounds binding to the sigma receptor generally also known as sigma ligands are well known in the art with many of them falling under the definition for “Compound/s binding to the sigma receptor” set up above. Still even though there are many uses known for sigma ligands such as antipsychotic drugs, anxiolytics, antidepressants, the treatment of stroke, antiepileptic drugs and many other indications including anti-migrane and general pain (mostly analgesia) there is nowhere any mentioning of these compounds being useful against allodynia.

Compounds which have an affinity to the sigma receptor known in the art are listed below. Some of these compounds do not only bind to sigma (and not al with high affinities) and so only part of these listed compounds do fall under the definition of “Compound/s binding to the sigma receptor” defined above, namely e.g. NE-100 and Haloperidol but also many others.

| | | |
|---|---|---|
| 3-PPP | Fluoexetine | Quetiapine |
| 8-OH0DPAT | Fluspirilene | Remoxipride |
| A-01 | Gevotroline | Repinotan |
| A-85380 | GR-218231 | RGH-1756 |
| Abaperidone | Granisetron | Rimcazole |
| ABT-089 | Haloperidol | Risperidone |
| ABT-702 | Harmaline | Ro-64-6198 |
| AC-915 | ICA-17043 | RS-102221 |
| AH-9700 | Ifenprodil | RS-67333 |
| Alniditan | Igmesine | RS-67506 |
| Altinicline | Iloperidone | S-15535 |
| Alvameline | Imipramine | S-33084 |
| Amantadine | L-687384 | S-33112 |
| Amiodarone | L-745870 | SA-4503 |
| Amisulpride | Lamotrigine | Safinamide |
| Amperozide | Lanepitant | Sertindole |
| Apomorphine | LEK-8829 | SH 3/28 |
| Aptiganel | LR-172 | SH-1/57 |
| Asenapine | LU-29253 | SH-2/21 |
| Astermizole | Mazaperitine | SH-3/24 |
| Atomoxetine | MCL-0129 | Siramesine |
| AZ-21666 | MDL-100907 | SK&F-10047 |
| Azasetron | MDL-28815 | SKF-10047 |
| Belaperidone | Memantine | SL-650155 |
| Benzoylecgonine | Metanicotine | Spiperone |
| Blonanserin | Metoclopramide | SR-31742A |
| BMY-14802 | Mizolastine | SSR-125329A |
| Bradyzide | MJ-139801 | SSR-240600 |
| Bromperidol | Mosapramine | Sulpiride |
| Buspirone | MR-22 | Sultopride |
| Carabersat | MS-377 | Sumatriptan |
| Chlorpromazine | Nafadotride | T-82 |
| Cilansetron | NAS-181 | Tacrine |
| Cisapride hydrate | NE-100 | Tamoxifen |
| Clomipramine | NE-535 | Tebanicline |
| Clorgyline | NE-537 | Terfenadine |
| Clozapine | Nemonapride | Testosterone |
| CNS-5161 | NGD-94-1 | Tiapride |
| Co-2-6749 | NNC-05-1869 | Tiospirone |
| Cocaine | NPC-16377 | Tolterodine |
| D-02 | NRA-0154 | Tranylcypromine |
| Deramciclane | NS-1209 | Trifluoperazine |
| Dextromethorphan | Ocaperidone | Vanoxerine |
| Dihydroergotamine | Olanzapine | Vilazodone |
| Ditolylguanidine | Ondansetron | XJ-448 |
| Dizocilpine | Opipramol | YM-50001 |
| Donepezil | Pargyline | YM-53389 |
| DuP-734 | PD-143188 | YM-57158 |
| Eltoprazine | PD-172760 | YZ-011 |
| FH-510 | Pentazocine | YZ-011 |
| | Perospirone | Zanapezil |
| | Phencyclidine | ZD-6021 |
| | Physostigmine | Ziprasidone |
| | Pipamperone | Zotepine |
| | Preclamol | |
| | Progesterone | |

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” is understood in particular, in the context of this invention, as salt (as defined above) formed either with a physiologically tolerated acid, that is to say salts of the particular active compound with inorganic or organic acids which are physiologically tolerated—especially if used on humans and/or mammals—or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, hydrobromide, monohydrobromide, monohydrochloride or hydrochloride, methiodide, methanesulfonic acid, fomic acid, acetic acid, oxalic acid, succinic acid, malic acid, tartaric acid, mandelic acid, fumaric acid, lactic acid, citric acid, glutamic acid, hippuric acid picric acid and/or aspartic acid. Examples of physiologically tolerated salts of particular bases are salts of alkali metals and alkaline earth metals and with NH₄.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

It is to be understood that the use according to the invention is restricted to allodynia in regards to all the pain types mentioned in here.

According to the IASP “allodynia” is defined as “a pain due to a stimulus which does not normally provoke pain” (IASP, Classification of chronic pain, 2^{nd} Edition, IASP Press (2002), 210). Even though the symptoms of allodynia are most likely associated as symptoms of neuropathic pain this is not necessarily the case so that there are symptoms of allodynia not connected to neuropathic pain though rendering allodynia in some areas brosder then neuropathic pain.

The IASP draws the following difference between “allodynia”, “hyperalgesia” and “hyperpathia” (IASP Classification of chronic pain, 2^{nd} Edition, IASP Press (2002), 212):

| | | |
|---|---|---|
| Allodynia | Lowered threshold | Stimulus and response |
| | | mode differ |
| Hyperalgesia | Increased response | Stimulus and response rate |
| | | are the same |
| Hyperpathia | Raised threshold; | Stimulus and response rate |
| | Increased response | may be the same or different |

In another preferred embodiment of the use according to the invention the allodynia, is central pain.

According to the IASP “central pain” is defined as “a pain initialed or caused by a primary lesion or dysfunction in the central nervous system” (IASP, Classification of chronic pain, 2^{nd} Edition, IASP Press (2002), 211).

In another embodiment of the use according to the invention the allodynia, is causalgia.

According to the IASP “causalgia” is defined as “a syndrome of sustained burning pain, allodynia and hyperpathia after a traumatic nerve lesion, often combined with vasomotor and sudomtor dysfunction and later trophic changes” (IASP, Classification of chronic pain, 2^{nd} Edition, IASP Press (2002), 210).

In another preferred embodiment of the use according to the invention the allodynia, is hyperesthesia.

According to the IASP “hyperesthesia” is defined as “increased sensitivity to stimulation, excluding the senses” (IASP, Classification of chronic pain, 2^{nd} Edition, IASP Press (2002),211).

In another preferred embodiment of the use according to the invention the allodynia, is neuralgia.

According to the IASP “neuralgia” is defined as “Pain in the distribution of a nerve or nerves” (IASP, Classification of chronic pain, 2^{nd} Edition, IASP Press (2002), 212).

In another preferred embodiment of the use according to the invention the allodynia, is neuritis.

According to the IASP “neuritis” is defined as “inflammation of a nerve or nerves” (IASP, Classification of chronic pain, 2^{nd} Edition, IASP Press (2002), 212).

In another preferred embodiment of the use according to the invention the allodynia, is neuropathy.

According to the IASP “neuritis” is defined as “a disturbance of function of pathological change in a nerve: in one nerve mononeuropathy, in several nerves mononeurpathy multiplex, if diffuse and bilateral, polyneuropathy” (IASP, Classification of chronic pain, 2^{nd} Edition, IASP Press (2002), 212).

In a very preferred embodiment of the invention the medicament is used for the treatment of allodynia in which the stimulus evoking the allodynia is mechanical.

In another embodiment of the invention the medicament is used for the treatment of allodynia in which the stimulus evoking the allodynia is thermal.

In a very preferred embodiment of the invention the compound binding to the sigma receptor used is acting on the sigma receptor as an antagonist.

In another embodiment of the invention the compound binding to the sigma receptor used is acting on the sigma receptor as an agonist.

In another embodiment of the invention the compound binding to the compound binding to the sigma receptor used is acting on the sigma receptor as a mixed agonist/antagonist, a partial agonist or a partial antagonist.

In a very preferred embodiment of the invention the sigma receptor to which the “compound binding to the sigma receptor” is binding to is the sigma 2 receptor. Under this embodiment “Compound/s binding to the sigma receptor” as used in this application is/are defined as having ≧95% displacement using 1 mM (₁H-pentazocine) and a Km Value in their binding to the sigma 2 receptor ≦50 nM (in regards to any one of the sigma receptor subtypes).

In human therapeutics, the dose administered can be quite low depending on the route of administration and is well known in the art because many sigma compounds are known therapeutics.

Any medicament according to the invention contains the active ingredient as well as optionally at least one auxiliary material and/or additive and/or optionally another active ingredient.

The auxiliary material and/or additive can be specifically selected from conserving agents emulsifiers and/or carriers for parental application. The selection of these auxiliary materials and/or additives and of the amounts to be used depends upon how the pharmaceutical composition is to be applied. Examples include here especially parental like intravenous subcutaneous or intramuscular application formulations but which could also be used for other administration routes.

Routes of administration can include intramuscular injection, intravenous injection, subcutaneous injection, sublingual, bucal, patch through skin, oral ingestion, implantable osmotic pump, collagen implants, aerosols or suppository.

Included in this invention are especially also methods of treatments of a patient or a mammal, including men, suffering from allodynia using compounds binding to the sigma receptor.

The examples and figures in the following section describing pharmacological trials are merely illustrative and the invention cannot be considered in any way as being restricted to these applications.

### FIGURES

FIG. 1) refers to example 1 and shows the test protocol for all tests with von Frey filaments.

FIG. 2a to c) refer to example 2 and show the effect of NE-100 a specific sigma receptor inhibitor in a model of allodynia, especially mechanical allodynia.

FIG. 2a) shows the dose dependency of the treatment with NE-100 to show analgesia in capsaicin-induced allodynia.

FIG. 2b) demonstrates that the treatment with NE-100 is effective specifically in mechanical allodynia and not general pain as shown by the different efficacy depending on the force of the von-Frey filaments with 0.5 g being typically in the range of allodynia and 4 g clearly being in the general pain field.

FIG. 2c) proofs that the effect of the treatment with NE-100 is clearly connected to its sigma inhibitor activity, as PRE-084 is a well known sigma receptor agonist.

FIGS. 3 a to d) refer to example 3 and shows the effect of antisense ODNs against sigma (1) receptor.

FIG. 3a) shows the test protocol for Oligodesoyxnucleotid (ODN) tests with von Frey filaments.

FIG. 3b) shoes the influence of the wash-out period on the effect of treatment with antisense ODN, with two known antisense ODN (by KING . . . and UEDA . . . ) being used proving their highly significant effect on allodynia in the von-Frey model. Still after 7 days washout the effect is gone as has to be expected from antisense ODN. Mismatches do not have significant effect.

FIG. 3c) shows the effectiveness and dose dependency with two known antisense ODNs (by KING and UEDA) testing with von Frey filaments. Mismatches do not have any significant effect.

FIG. 3d) demonstrates that the treatment with two known antisense ODNs is effective specifically in the mechanical allodynia and not general pain as shown by the different efficacy depending on the force of the von-Frey filaments with 0.5 g being typically in the range of allodynia and 4 g clearly being in the general pain field.

FIG. 4) refers to example 4 and demonstrates clearly that KO-Mice not having the sigma (1) receptor (called “mutantes”) are not susceptible to the allodynia-induced effects of capsaicin independent of the dose given compared to wild-type mice (called “salvajes”). This is clearly demonstrating the truth of the role of sigma receptors in allodynia and strengthens the claim to the role of all compounds binding to the sigma-receptor in allodynia.

### EXAMPLES

### Example 1

### Von Frey-Model

The von Frey model is a model for allodynia, stimulated mechanically.

Interest of the model:
The injection of capsaicin to experimental animals produces acute pain followed by allodynia
The mechanisms involved in capsaicin-induced acute pain and allodynia are relatively well known (mainly activation of peripheral nociceptors and sensitization of spinal cord neurons, respectively)
   Hypothesis
Capsaicin-induced allodynia is due to the release in the spinal cord of several substances including excitatory aminoacids (EA). Since sigma ligands modulate the effect of EA they should also modulate capsaicin-induced allodynia.

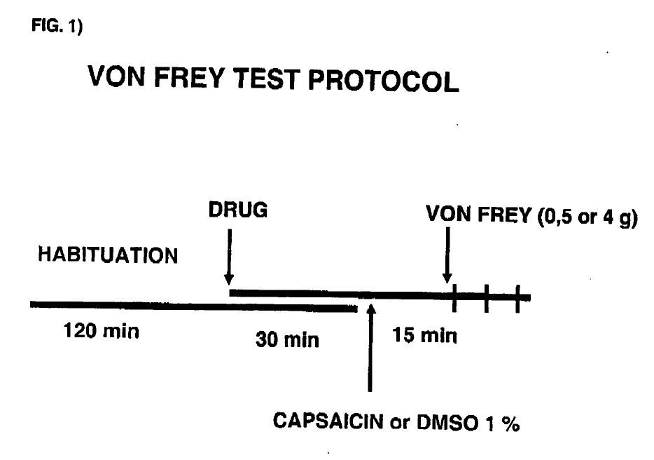

FIG. 1) shows the test protocol for all tests with von Frey filaments. After habitation mice were according to FIG. 1 first treated with the test-compound (or not in controls). Then capsaicin (1% DMSO) is injected into their paw resulting in developing pain in the effected paw. The effected paw is then treated with a mechanical stimulus and the latency time before the paw is withdrawn is measured.

### Example 2

### Effect of NE-100 in the Von Frey-Model

NE-100 is a well known compound with high affinity to the sigma receptor, more specifically a known specific inhibitor of Sigma 1. This pharmacological test showed the effect of NE-100 a specific sigma receptor inhibitor in the von-frey model described in example 1, a model of allodynia.

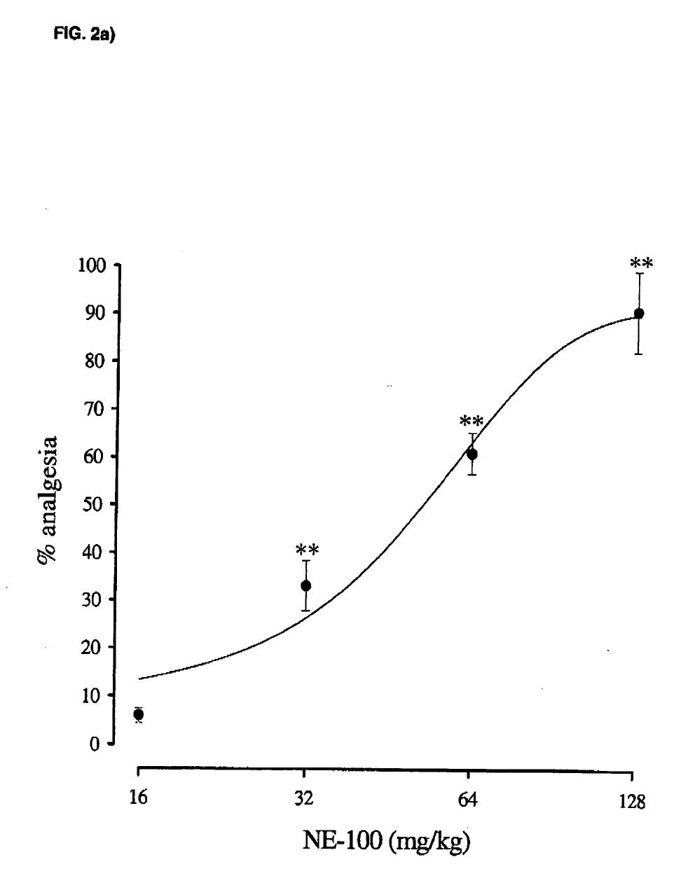

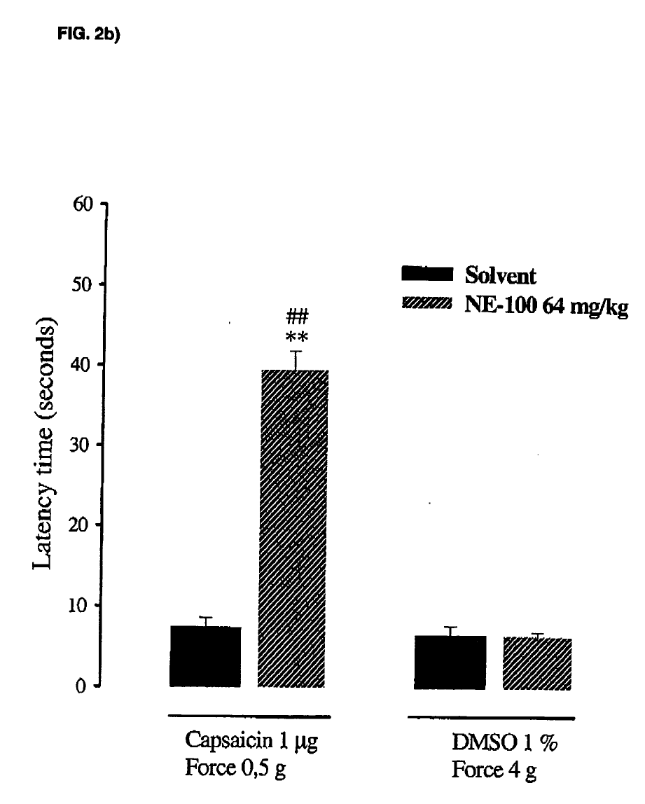

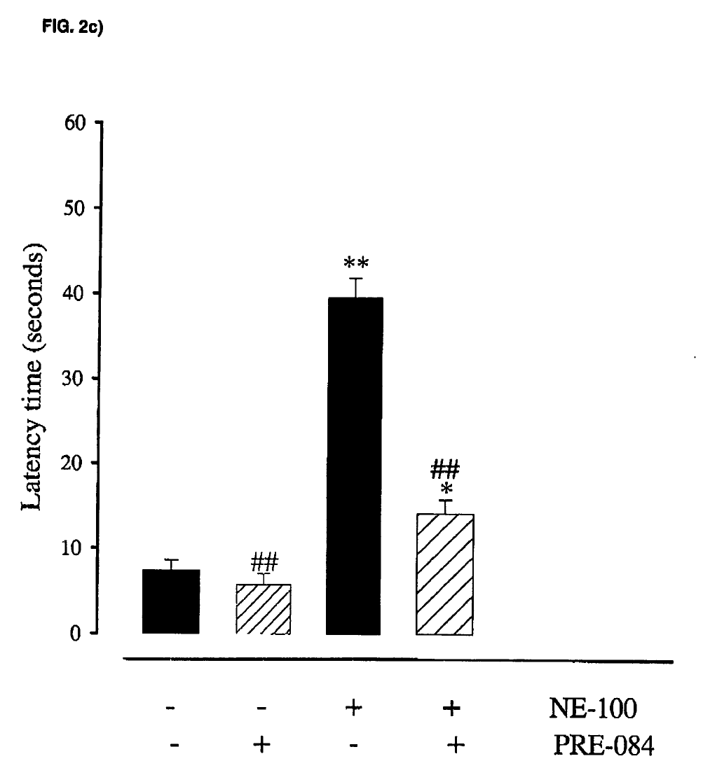

As shown in FIG. 2a) there is a dose dependency of the treatment with NE-100 showing analgesia in capsaicin-induced allodynia.

As demonstrated in FIG. 2b) the treatment with NE-100 is effective specifically in allodynia or mechanical allodynia and not general pain as shown by the different efficacy depending on the force of the von-Frey filaments with 0.5 g being typically in the range of allodynia and 4 g clearly being in the general pain field.

Further as shown in FIG. 2c) there is clear evidence that the effect of the treatment with NE-100 is clearly connected to its sigma inhibitor activity, as PRE-084 is a well known sigma receptor agonist counteracting the effect of NE-100.

### Example 3

### Effect of Antisense ODN Against the Sigma Receptor in the Von Frey-Model

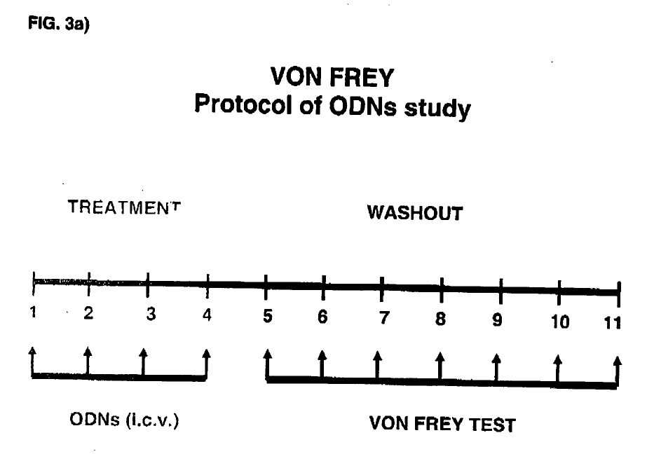

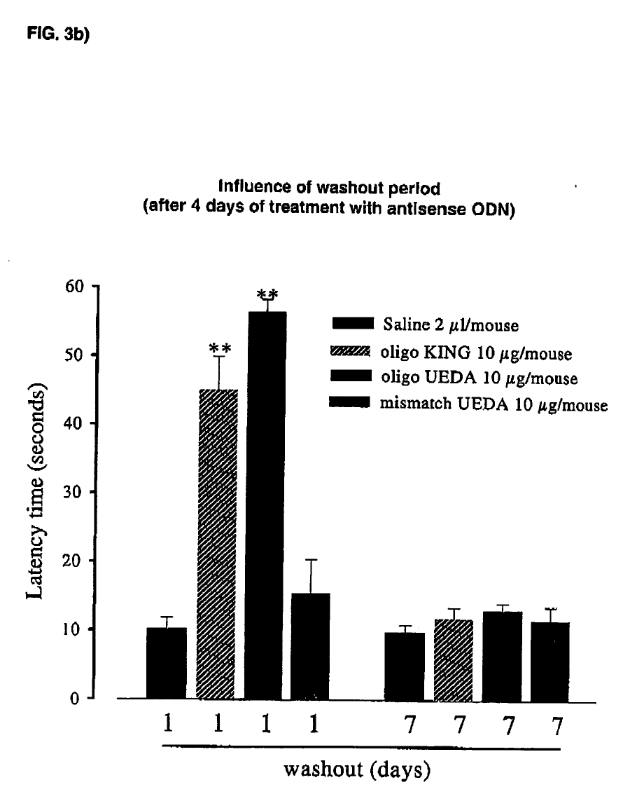

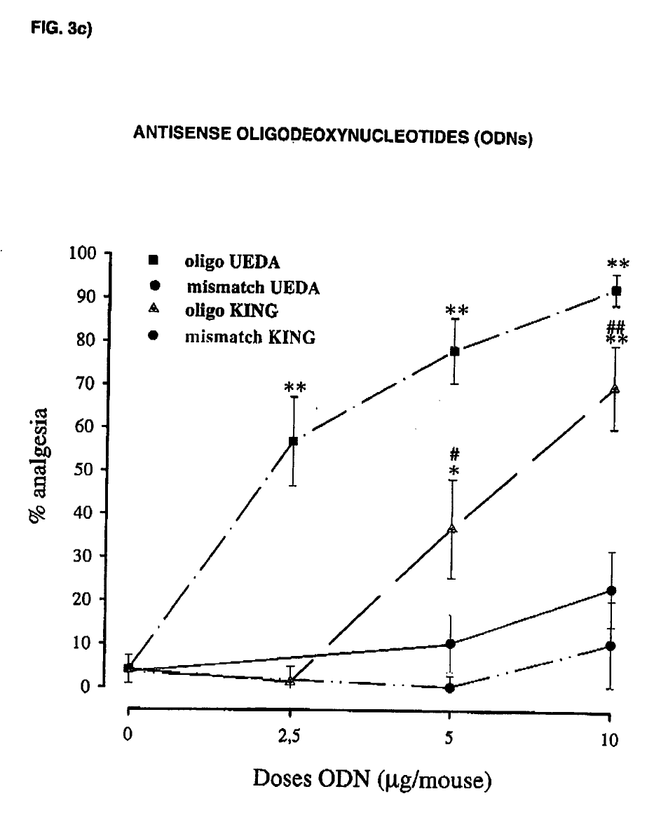

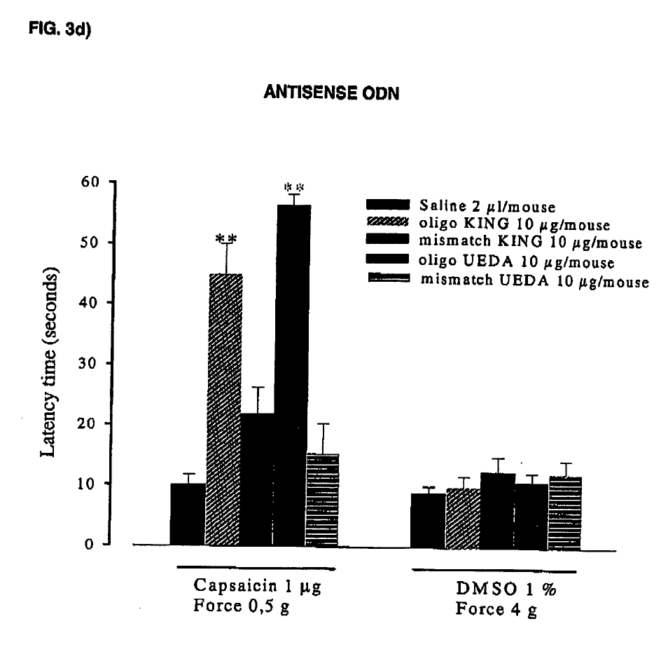

2 well known antisense Oligodesoxynucleotides (ODN) against the sigma 1 receptor (KING et al . . . and UEDA et al . . . ) were synthesized and according to the protocol shown in FIG. 3a) given on 4 consecutive days i.c.v. followed by a wash-out period and von-Frey tests according to example 1.

As can be seen in FIG. 3b) both antisense ODNs show a strong effect on day one after treatment with mismatches not having any significant effect. This effect is washed out after 7 days as can be expected from antisense ODN.

The effectiveness and dose dependency is demonstrated with FIG. 3c). Mismatches do not have any significant effect.

Further as demonstrated in FIG. 3d) the treatment with the two known antisense ODNs is effective specifically in allodynia or mechanical allodynia and not general pain as shown by the different efficacy depending on the force of the von-Frey filaments with 0.5 g being typically in the range of allodynia and 4 g clearly being in the general pain field.

### Example 4

### Effect of the Von Frey-Model on KO Mice

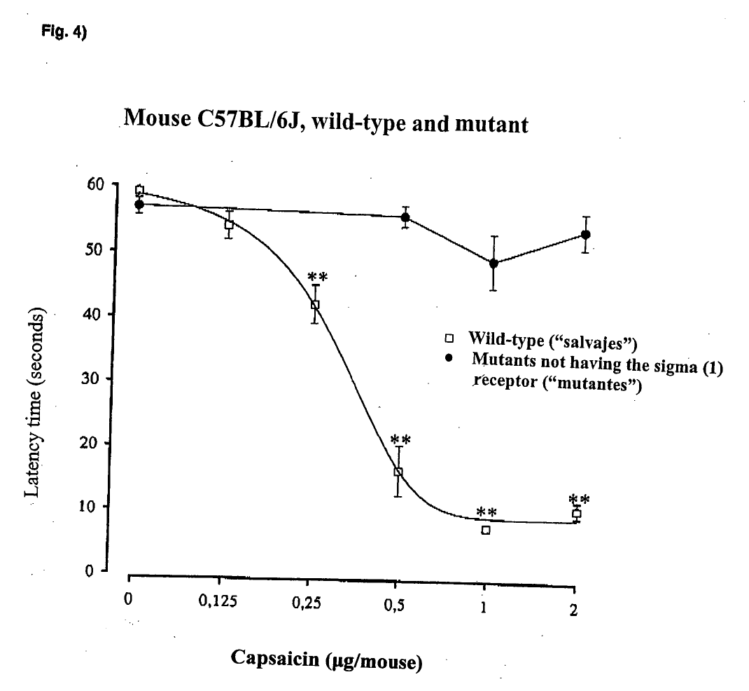

KO mice lacking the sigma 1 receptor were prepared according to WO 2004/52092 and tested in comparison to wild-type mice in the von-Frey model. As demonstrated in FIG. 4) KO-Mice hot having the sigma (1) receptor (called “mutantes”) are not susceptible anymore to the allodynia inducing effects of capsaicin independent of the dose given compared to wild-type mice (called “salvajes”). This is clearly demonstrating the truth of the role of sigma receptors in allodynia and strengthens the claim to the role of all compounds binding to the sigma-receptor in allodynia.

1. A method of treating allodynia, the method comprising administering a compound that binds to a sigma receptor.

2. The method of claim 1, wherein the allodynia is central pain.

3. The method of claim 1, wherein the allodynia is peripheral pain.

4. The method of claim 1, wherein the allodynia is causalgia.

5. The method of claim 1, wherein the allodynia is hyperesthesia.

6. The method of claim 1, wherein the allodynia is neuralgia.

7. The method of claim 1, wherein the allodynia is neuritis.

8. The method of claim 1, wherein the allodynia is neuropathy.

9. The method of claim 2, wherein the allodynia is evoked by a mechanical stimulus.

10. The method of claim 2, wherein the allodynia is evoked by a thermal stimulus.

11. The method of claim 1, wherein the compound that binds to the sigma receptor is an antagonist of the sigma receptor.

12. The method of claim 1, wherein the compound that binds to the sigma receptor is an agonist of the sigma receptor.

13. The method of claim 1, wherein the compound that binds to the sigma receptor is (i) a mixed agonist/antagonist, (ii) a partial agonist, or (iii) a partial antagonist, of the sigma receptor.

14. The method of claim 1, wherein the sigma receptor is a sigma 1 receptor.

15. The method of claim 1, wherein the sigma is a sigma 2 receptor.

16. The method of claim 3, wherein the allodynia is evoked by a mechanical stimulus.

17. The method of claim 3, wherein the allodynia is evoked by a thermal stimulus.
